(19) United States
(12) Patent Application Publication
(10) Pub. No.: US 2003/0225445 A1
(43) Pub. Date: Dec. 4, 2003

(54) Surgical stent delivery devices and methods

(76) Inventors: Patricia M. Derus, Rogers, MN (US); Steven C. Christian, New Brighton, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/438,761

(22) Filed: May 14, 2003

Related U.S. Application Data
(60) Provisional application No. 60/380,804, filed on May 14, 2002.

(51) Int. Cl. A61F 2/06

(52) U.S. Cl. 623/1.11

(57) ABSTRACT

A device for loading a self-expanding stent into a receiving area of a delivery tool is provided, the device comprising a stent storage region having an internal chamber sized to retain the stent in a first state, a stent transfer region spaced from the storage region and having an internal chamber sized to receive, retain, and transfer the self-expanding stent in a second state in which the stent is at least partially compressed relative to the first state of the stent, a stent compressing region extending between the storage region and the transfer region and having an internal chamber for radially collapsing the stent from the first state to a second state, and a loading mechanism slideably positioned relative to the transfer region for slideably moving the stent in the second state from the transfer region toward the compressing region.

This application claims the benefit of U.S. Provisional application Serial No. 60/380,804, filed May 14, 2002, entitled “SURGICAL STENT DEVICES AND METHODS,” which application is incorporated herein by reference in its entirety.

### TECHNICAL FIELD

The present invention relates generally to devices and methods used for loading a stent into a delivery tool for placement in a body cavity. More specifically, the present invention is directed to devices and methods for storing a self-expanding stent until the approximate time of surgical placement, then loading the stent in a more compressed state into a delivery tool for controlled stent deployment.

### BACKGROUND OF THE INVENTION

Tubular prosthetic devices for transluminal implantation in body canals, such as the urethra or blood vessels, for the purpose of repair or dilation are known in the art. These prosthetic devices are commonly known as stents, and can include the types of stents that are self-expanding in the radial direction with a reduction of compression force on the outer walls of the stent. One typical self-expanding stent is disclosed, for example, in U.S. Pat. No. 4,665,771 (Wallsten), which includes a radially and axially flexible, elastic tubular body of a predetermined diameter that is variable under axial movement of the ends of the body relative to each other.

Self-expanding stents are typically used in applications where it is desirable to minimize acute and chronic trauma to the luminal wall when implanting an intraluminal stent. Thus, a stent that applies a gentle radial force against the wall, and that is compliant and flexible when subjected to lumen movements, is preferred for use in diseased, weakened, or brittle lumens. Preferably, a stent is further capable of withstanding radially occlusive pressure from tumors, plaque, tissue hypertrophy and luminal recoil, and remodeling.

A delivery tool that retains the stent in its radially compressed state is often used to present the stent to a treatment site within the body, where the flexible nature and reduced radius of the radially compressed stent enables delivery to the treatment site through relatively small and curved tracts, lumens, or vessels. In such deliveries of stents, the delivery tool is typically inserted into a body opening and passed through the various body vessels to the treatment site. After the stent is properly positioned at the treatment site, the delivery tool is actuated to release the positioned stent. With the compressive force of the delivery tool removed, the stent is then able to expand within the body vessel. The delivery tool may then be removed from the body, while the stent remains in the vessel at the treatment site as an implant. Typically, the delivery tool is designed for single use, so it may be discarded after the stent is delivered.

Stents can generally be grouped into the three categories of metal stents, fenestrated or laser-cut polymeric stents, and radially expanding polymeric stents. Metal stents do not degrade within the body and are thus typically used in applications where a permanent stent is desired. Metal stents are further advantageous in that they do not experience significant creep or plastic deformation when stored in a compressed state for an extended period of time. Although metal stents can provide a permanent solution for the patient, if such stents need to be removed for any reason, such as in cases where the metal stent loses some of its strength and/or if the patient needs other treatments in the same area of the body, the metal stents can only be removed with additional surgery.

Fenestrated stents are typically formed by providing a solid tube of polymeric stent material and using a laser to cut a pattern of holes through the wall of the tube in order to create a mesh-like appearance. Fenestrated stents are often very expensive, however, due to the large amount of polymeric material that is wasted in the laser cutting process and the time-consuming process of precisely cutting the tube with a laser to create a desired pattern. These stents are often further inappropriate for a particular application due to the tendency of fenestrated stents to resist radial expansion and longitudinal compression.

Radially expanding polymeric stents are typically used to artificially and temporarily keep a lumen open, but due to the material from which the polymeric stents are made, these stents begin to degrade when exposed to water and are thus eventually excreted or sloughed off by the body. This degradation is typically predictable based on the material used, thereby providing an excellent means for keeping a lumen open for a relatively short, predetermined period of time, while eliminating the need to surgically remove the stent when it is no longer needed. However, polymeric stents can experience creep when deformed or compressed for a period of time, and may thus become useless if stored in a compressed state for an extended time period. In other words, polymeric stents that are stored in a compressed state for a long period of time will often lose their ability to radially expand back to a size that is useful to support a body cavity.

In addition to the problems associated with creep or permanent polymeric stent deformation described above, polymeric stents are susceptible to absorbing water from their surrounding environment and becoming contaminated if not properly dry-sealed in an impermeable packaging material. The packaging materials and processes involved in keeping the components and any surrounding air within the package completely dry can be expensive and time-consuming, which is particularly true for dry-sealing a relatively large device like a delivery tool having a loaded stent. Thus, when a polymeric stent that is prone to absorbing moisture is loaded into a delivery tool, the entire delivery tool including the loaded polymeric stent must be dry-sealed, even though only the stent portion of the device actually needs the extremely dry packaging. It would therefore be advantageous to package each polymeric stent separate from its delivery tool so that the stent can be loaded into the delivery tool at the approximate time the stent is to be inserted into a patient. In this type of arrangement, only the stent and its packaging need to be subjected to the expensive environmental controls required to keep the stent dry.

With any of the various types of stents described above, a delivery tool of some type is needed to precisely insert the stent into a body lumen of a patient. Many delivery tools are available in the art, where the delivery tools often have a handle assembly that includes a main body with a fixed rear loop handle and a moveable forward loop handle of the type described, for example, in U.S. Patent Publication No. US 2002/0183827 (Derus et al.). To deploy a stent from using this type of handle assembly, the surgeon would typically place a thumb in the rear loop handle and a finger in the forward loop handle, then pull the forward loop handle toward the rear loop handle by squeezing the inserted finger toward the thumb. While handles of this type often provide precise positioning of a stent, there is a continued need for different types of delivery tools to accommodate the preferences of a wide variety of surgeons.

### SUMMARY OF THE INVENTION

The present invention is directed to methods and devices by which a stent may be loaded into a delivery tool prior to its implantation within a patient. In particular, the present invention is directed to the radial compression of a self-expanding stent to reduce its diameter from its storage state, then transferring the compressed stent into a delivery tool. Drawing the stent from a storage area or chamber of a cartridge through a funnel section and into a transfer or loading chamber that is smaller than the storage chamber preferably accomplishes the radial compression of the stent. In one aspect of the invention, one or more sutures or threads may be attached to the stent and positioned so that the threads extend through the body of the cartridge and are accessible to the person desiring to load the stent into a delivery tool. The stent is then pulled, preferably by the attached threads, from the storage area to the transfer chamber until the stent is generally enclosed within the transfer chamber. After the stent is in this transfer chamber, the threads can be cut, if desired, then the delivery tool can be introduced into an opening in, and generally concentric with, the end of the cartridge where the stent had previously been stored. When the delivery tool is properly positioned within the cartridge, a plunger or other transfer mechanism is activated to drive the stent into the delivery tool. At this point, the delivery tool can be removed from the cartridge and used to implant the stent in a body cavity.

The devices and methods of the present invention offer a number of advantages over certain prior art systems. For example, the present invention provides devices, methods, and systems that require relatively few, uncomplicated components that can accommodate a wide range of stent sizes. The cartridges may function independently of a delivery system and may be used multiple times, if desired. Further, when a degradable stent is used, the methods and devices of the present system require a relatively small amount of dry storage volume as compared to systems that use dry storage for the delivery tool and stent in combination.

The present invention further provides a handle design that provides a mechanism for a stent delivery tool that improves the mechanics of the delivery system, simplifies the design, is usable for a wide range of stent sizes, and promotes smooth, consistent, and controlled operation. In particular, the invention provides a delivery tool having a handle that incorporates a slider linkage, such as a 4-bar slider linkage, where a pivot point of the linkage is located on the axis of a concentric inner and outer tubing of the delivery tool. In this way, the moment load to these components may be minimized, and the potential for binding to occur between the components is also reduced. These features can be beneficial to a surgeon by providing a tool with improved tactile feedback, better placement precision, and reduced time and difficulty in deploying the stent. With regard to manufacturability of the delivery tool, the present handle requires relatively small assembly time and uses a small number of parts. The handle design is further advantageous in that it is adaptable to multiple linkage configurations and locking systems, where the ability to change linkage configurations can make the basic design applicable to other applications. The tool may further include a locking system to prevent and allow relative movement of the components during the various stent loading and deployment or implantation processes, as desired. The handle design may instead include a V-shaped handle and/or may include springs or other mechanisms that allow precise handling and placement by a surgeon during an implantation process.

In one aspect of this invention, a device is provided loading a self-expanding stent into a receiving area of a delivery tool. The device comprises a stent storage region having an internal chamber sized to retain the stent in a first state, a stent transfer region spaced from the storage region and having an internal chamber sized to receive, retain, and transfer the self-expanding stent in a second state in which the stent is at least partially compressed relative to the first state of the stent, and a stent compressing region extending between the storage region and the transfer region and having an internal chamber for radially collapsing the stent from the first state to a second state. The device further comprises a loading mechanism slideably positioned relative to the transfer region for slideably moving the stent in the second state from the transfer region toward the compressing region.

The device may further comprise a stent transfer mechanism for moving the stent from the storage region to the transfer region through the compression region while compressing the stent from the first state to the second state, wherein the stent transfer mechanism may include an extending member that is moveable from the storage region to the transfer region. The stent transfer mechanism is may be attached to a portion of the stent in such a way that it can be removed. The extending member of the stent transfer mechanism may be a generally flexible material and may specifically be made of a thread-like material. The loading mechanism of the device may include an extending portion having a distal end that is engageable with a portion of the stent to slideably force the stent in the second state from the transfer region toward the compressing region. The device may further comprise a spool insert positioned at least partially within the internal chamber of the storage region, wherein the spool insert is sized to support an interior portion of the stent provided in the first state.

The device described above may further be provided in combination with a self-expanding stent provided in the first state within the internal chamber of the storage region, wherein the stent comprises a substantially tubular, radially expandable body portion. The device described above may also be provided in combination with a delivery tool comprising an outer tube and an inner tube, wherein the outer tube is slideable relative to the inner tube, and further comprising a receiving area inside the outer tube and adjacent a first end of the outer tube, wherein the outer tube of the delivery tool may be sized to be inserted into the internal chamber of the storage region and the internal chamber of the compressing region of the device.

In another aspect of the invention a method is provided of loading a self-expanding stent into the receiving area of a loading tool, the method comprising the steps of providing a stent cartridge comprising a stent storage region having an internal chamber including a stent in a first state, a stent transfer region spaced from the storage region and having an internal chamber sized to receive, retain, and transfer the self-expanding stent in a second state in which the stent is at least partially compressed relative to the first state of the stent, a stent compressing region having an internal chamber extending between the storage region and the transfer region, and a loading mechanism slideably positioned relative to the transfer region. The method further comprises moving the stent from the internal chamber of the storage region through the internal chamber of the compressing region to the internal chamber of the transfer region, wherein the stent is compressed to the second state, then inserting a first end of a delivery tool through an end of the stent storage region and into the stent compressing region, the delivery tool comprising an outer tube having a first end and an inner tube positioned within the outer tube, wherein the outer tube has a receiving area adjacent to the first end, and wherein the inner tube is slideable relative to the outer tube. The method also includes transferring the stent in the second state from the transfer region of the cartridge into the receiving area of the delivery tool by moving the loading mechanism toward the compressing region.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of a stent of the type that can be used in conjunction with the methods, devices, and systems of the present invention;

FIG. 2 is a cross-sectional side view of a stent cartridge or device of the present invention, including a stent in its expanded state within a storage region of the device;

FIG. 3 is a cross-sectional side view of the stent cartridge of FIG. 2, with the further inclusion of a portion of a delivery tool inserted therein, and including a loading mechanism in a retracted state and a stent in its compressed state;

FIG. 4 is a cross-sectional side view of the stent cartridge of FIGS. 2 and 3, with the stent inserted into the end of the delivery tool;

FIG. 5 is a cross-sectional side view of an alternative stent cartridge in accordance with the present invention;

FIG. 6 is a side view of one embodiment of a delivery tool for stent placement and delivery;

FIG. 7 is perspective view of another embodiment of a delivery tool for stent placement and delivery;

FIG. 8 is a perspective view of another embodiment of a delivery tool for stent placement and delivery; and

FIG. 9 is a side view of a delivery system for stents including a stent cartridge and a loading tool similar to that illustrated in FIG. 6.

### DETAILED DESCRIPTION OF THE PREFERRED EMBODIMENTS

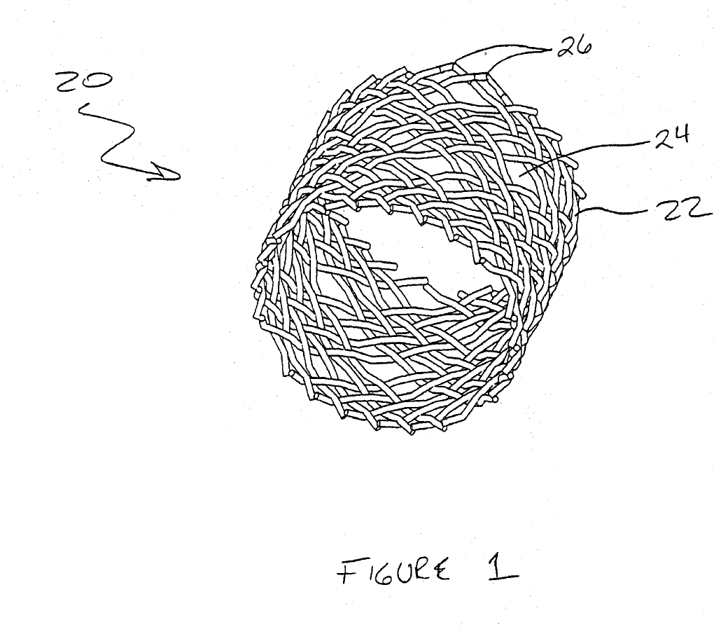

Referring now to the Figures, wherein the components are labeled with like numerals throughout the several Figures, and initially to FIG. 1, one preferred configuration of a stent 20 in accordance with the present invention is illustrated. This exemplary stent or prosthetic device 20 has a flexible, generally tubular shape and generally comprises a plurality of interwoven or interconnected fibers or wires 22 defining a series of interconnected cells or openings 24 between the fibers 22. The fibers 22 may be arranged so that one or both ends of stent 20 include a series of loops 26 around the periphery of the stent or may include a series of loose fiber ends. It is further preferable that the stent 20 is of the type generally known as a self-expanding stent. The length of the stent 20 may range from about 10 mm to about 500 mm, preferably from about 10 mm to about 50 mm, with the relaxed diameter ranging from about 4 mm to about 45 mm, preferably from about 5 mm to about 25 mm.

Self-expanding stents, as referred to herein, are devices that can be radially compressed from their relaxed state when subjected to external forces, but can spring or expand at least partially back toward their relaxed state when the external forces are removed or reduced. In particular, self-expanding stents are typically devices that are either initially provided with a relatively low cross-sectional profile or radius, or are compressed to such a cross-sectional profile, for insertion into a small tubular body lumen, such as a urethra, for example. This lower cross-sectional profile can be particularly advantageous in situations where it is necessary to deliver the stent through relatively small and curved tracts, lumens, or vessels. When the stent is inserted and properly positioned within the body, the external forces on the stent may be removed to allow it to expand to a larger diameter, thereby providing the necessary support to the lumen in which it is inserted. Due to the geometry of the stent, the stent will typically be longer when in its compressed state and shorter when in its expanded state. Thus, devices that are similar to that of stent 20 are preferably made of materials that will expand to predictable dimensions upon their insertion into a body organ, and then retain those same dimensions for an extended period of time to continuously provide the desired support for the body organ, such as to prevent that body organ from collapsing or constricting. That is, the stents used with the present invention are preferably made of materials having a high modulus of elasticity so that they can be compressed to a relatively small diameter when subjected to various forces that cause it to constrict, then spring back a specified amount when the various forces are removed or reduced.

While the devices and methods of the present invention are applicable to a wide variety of stent configurations and materials, the present invention provides particular advantages for implantation of stents that permanently or plastically deform to some degree when subjected to external forces for a period of time, such as when compressed. With materials of this type, it is often advantageous to minimize the amount of time that the stents are compressed to reduce the probability of the stent taking a “set” or permanently deforming to such a degree that it cannot sufficiently expand back toward its original, uncompressed state when the external forces are removed. Such stent materials include, for example, bioresorbable, biocompatible polymeric materials such as hompolymers, copolymers, and blends of two or more homopolymers or copolymers, optionally including additives. A polymeric, bioresorbable stent may be of any stent configuration, such as a stent comprising woven, extruded monofilaments, or a stent comprising an extruded and cut fenestrated, self-expanding stent. Examples of bioresorbable stents and their materials and methods of their manufacture can be found, for example, in U.S. Pat. No. 6,368,346, the disclosure of which is incorporaed herein by reference. Exemplary polymeric materials include poly-L-lactic acid (PLLA), poly-D,L-lactic acid (PDLA), poly-e-caprolactone (PCL), and similar polymers. One preferred polymeric material for a bio-resorbable, biocompatible stent can be a blend of PLLA and PCL, e.g., in a ratio of 80:20 to 99:1 (PLLA:PCL). It is contemplated, however, that the devices and methods of the present invention are equally adaptable to stents made of materials that do not experience any appreciable creep or permanent deformation when stored in a compressed state for extended periods of time, such as stents made from a plurality of interwoven metal fibers or wires.

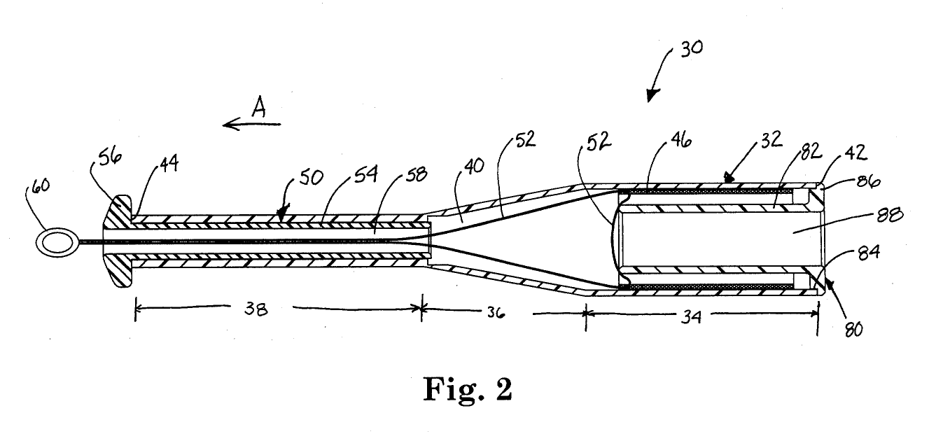

Referring now to FIG. 2, one preferred embodiment of a stent cartridge or device 30 is shown, which generally includes a body 32 having a stent storage region 34, a stent compressing region 36, and a stent transfer region 38. The device 30 includes an internal chamber 40 that extends from a proximal end 42 of the body 32 adjacent the storage region 34 to a distal end 44 adjacent the transfer region 38. Device 30 is further shown with a stent 46 in its uncompressed state within the storage region 34. As will be described in further detail below, this stent 46 can be moved between the various regions of the body 32 to provide the desired stent compression and loading into a delivery tool in accordance with the present invention. In particular, the internal chamber 40 of the storage region 34 is preferably sized to be large enough to retain a particular stent in its natural or uncompressed state without any unnecessary or undesired compressive forces being applied to the outside of the stent. It is contemplated, however, that the stent 46 may be partially compressed when in the storage region 34. Further, the storage region 34 should extend far enough in the longitudinal direction of the device 30 to retain the entire length of a stent being stored therein. That is, when a stent is being stored within the device 30, it will preferably not extend into the compressing region 36 or any part of the body 32 in which the stent would be subjected to compressive forces that cause permanent stent deformation and will instead be contained within the storage region 34.

As shown, the stent compressing region 36 extends between the storage region 34 and the transfer region 38, and is generally funnel-shaped such that the compressing region 36, as shown, is shaped as a truncated conical section. In this embodiment, it is not necessary that the compressing region 36 is long enough to retain the entire length of a stent 46 at any particular time; however, it is preferable that the compression region 36 is sufficiently long that the radius reduction of the internal chamber 40 in this section is relatively gradual. In particular, the compression region 36 is preferably provided with sufficient length that stent 46 can move easily through the device 30 while being subjected to relatively gradual compressive forces that do not undesirably deform or otherwise damage the stent. In any case, the internal chamber 40 of the compression region 36 is sized for radially collapsing the stent 46 from an expanded state to the compressed or partially compressed state in which it will be held within the transfer region 38. It is preferable that the walls of the compressing region 36 taper at a constant angle from the storage region 34 to the transfer region 38 to provide a consistent reduction of the internal radius of the internal chamber 40 in the compressing region 36. However, the reduction of the internal radius of the compressing region 34 may instead include a variety of tapered sections, where some sections include a different slope or taper than other sections within this region. Alternatively, stepped or less consistent dimensional changes of the compression region 34 may be provided.

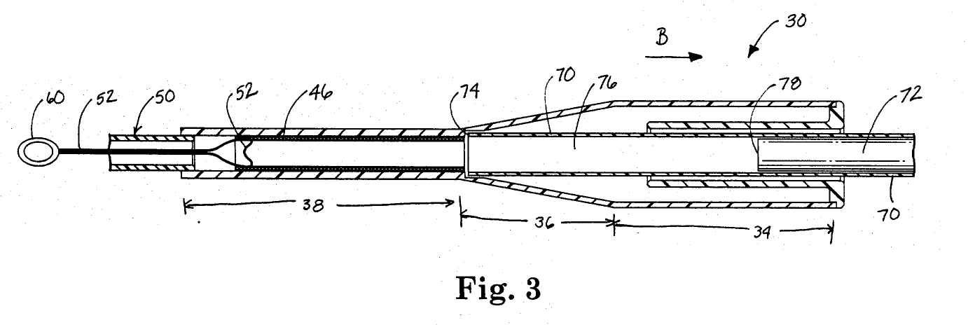

Stent 40 is preferably provided with at least one transfer mechanism, such as one or more threads or sutures 52 of the type illustrated in FIGS. 2 and 3, for facilitating the transfer of the stent 46 from the storage area 34 to the transfer area 38. The threads 52 can be attached to the stent 46 in any number of ways, such as with adhesives, hooks, or the like. In one preferred embodiment of the invention, the thread or threads 52 are inserted through one or more openings in the stent 46, such as the openings 24 of the stent shown in FIG. 1, or such as the loops 26 at one end of the stent when such loops are provided. The threads 52 can be attached to the stent at any point or points along the length of the stent that allow the stent to be stretched or pulled without breaking or substantially deforming the fibers of the stent. Thus, if the force required to pull the stent is higher than the force that the stent fibers near the end of the stent can withstand without breaking or unraveling, the thread 52 should be attached to the stent at a point or points that are spaced some further distance from the ends of the stent. Preferably, the threads 52 are attached as close to the end of the stent that will be pulled as possible to cause extension of the stent while it is being compressed through the compressing region.

In one arrangement, a single thread 52 is attached to a stent 46, wherein a thread 52 is inserted through a hole or opening in one side of the stent 46 at a point that is near the end of the stent 46 that will be pulled, then extended across the center of the stent 46 to approximately the diametrically opposite side of the stent 46. The thread 52 can then be inserted through a hole in this side of the stent 46 and pulled through the stent 46 until the extending portion of the thread 52 is long enough to be used for transfer of the stent 46 within the device 30 in accordance with the methods of the present invention. In many cases, a single thread 52 will be sufficient to accomplish the necessary transfer of the stent. However, additional threads 52 can be similarly attached at various points around the stent, if desired, to better distribute the forces that will be imparted to the stent when it is pulled. In a configuration of this type, the threads 52 may cross over each other in the center area of the stent or may not cross each other if multiple threads are inserted in holes in the stent that are not diametrically opposite each other. It is also contemplated that a single thread 52 may be used that is woven through multiple holes or loops around the perimeter of the stent, which can also serve to distribute the forces to multiple fibers or wires of the stent when it is pulled to further minimize the possibility of stent fiber breakage and/or deformation. The material from which the threads or sutures 52 are made should be sufficiently strong that they will not break or stretch when pulling the stent from the storage region 34 to the transfer region 38.

With further reference to FIG. 2, when the stent 46 is loaded or stored within the storage region 34 of the device 30, the thread or threads 52 attached to the stent 46 are positioned to extend through the compression region 36 and transfer region 38 and beyond the distal end 44 of the device 30. To move the stent 46 into the transfer region 38, the threads 52 extending from the distal end 44 are pulled in a direction A to move the stent through the compression region 36 and into the transfer region 38. Referring also to FIG. 3, the transfer region 38 is preferably sized to receive and retain a stent 46, such as a self-expanding stent, in a relatively compressed state in which the stent has a smaller radius as compared to the expanded or uncompressed state of the stent. In particular, FIG. 3 shows the stent 46 in a relatively compressed state and extending only within the transfer region 38. As with the storage region 34, the transfer region 38 preferably extends far enough in the longitudinal direction of the device 30 to contain the entire length of a stent in its compressed state when being held therein, as shown in FIG. 3. That is, when a stent is compressed to a predetermined size and is moved into the transfer region 38, it will preferably not extend into the compressing region 36 such that the entire stent is retained within the transfer region 38 in its compressed state. Although it is possible that a portion of the stent extends beyond the transfer region 38, any portion of the stent that extends into the compression region 36 will tend to expand to be a larger diameter than the portion of the stent within the transfer region, which may complicate the process of loading the stent into a delivery tool.

The transfer region 38 is preferably also provided with a loading member or plunger 50 positioned therein that is moveable or slideable in the longitudinal direction of the device 30. In this embodiment of the invention, the loading member 50 includes an elongated body portion 54 and a cap 56 at one end of the body portion 54. The body portion 54 and cap 56 are preferably provided with an elongated opening 58 extending from one end of the loading member 50 to the other. The body portion 54 is also preferably sized so that the loading member 50 can slide relatively easily with respect to the transfer region 38 in which it is provided. In particular, the loading member 50 can be positioned within the transfer region 38 when the stent 46 is in the storage region 34, but can preferably move a sufficient distance out of the storage region 34, in direction A, to provide enough space to accommodate the entire stent 46 in its compressed state within the region 38. The loading member 50 is also moveable back into the transfer region 38 by applying sufficient force on the cap 56 to push the stent 46 toward the compression region 36, when desired. Thus, this transfer region 38 is designed and sized to receive, retain, and transfer a stent in its compressed or partially compressed condition. In any case, the inside chamber 40 of the transfer region 38 preferably has cross-sectional dimensions that are equal to or slightly smaller than the internal dimensions of a loading tool or device into which the stent will be loaded, the advantages of which will be described in further detail below. In addition, the inside of the transfer region 38 near the distal end 44 and/or the outside of the body portion 54 may be provided with a shoulder portion or other configuration that acts as a stop to keep the loading member 50 from being pulled completely from the end 44 of the device 30. When such a configuration is used, the loading member 50 should preferably be able to move out of the transfer region 38 a sufficient distance to allow containment of a compressed stent therein.

As described above, moving the stent 46 from the storage region 34 to the transfer region 38 is preferably accomplished by pulling threads 52 attached to the stent. These threads are not necessarily attached to any other components of the device 30; however, the threads can be attached to the loading member 50 so that pulling the cap 56 of member 50 will simultaneously pull the threads 52 attached to the stent 46 within the device. In one embodiment of the invention, the elongated opening 58 in the body portion 54 is sized to accommodate one or more threads passing from one end of the loading member 50 to the other. In this arrangement, the threads 52 can extend from the stent 46 in the storage region 34, through the compressing region 36, through the entire length of the opening 58 of the loading member 50, and then extend beyond the distal end 44 of the device 30.

The motion of the threads 52 pulling the stent 46 and the motion of the loading member 50 sliding out of the transfer region 38 may occur simultaneously or may be independent operations. For example, if the threads 52 are not attached to the loading member 50, the loading member 50 may first be retracted from the transfer region 38 by a distance to allow sufficient space to contain a compressed stent, then the threads 52 attached to the stent 46 may be pulled to move the stent 46 into the transfer region 38. In another example of an arrangement where the threads are not attached to the loading device 50, the threads 52 that extend through the opening 58 can be pulled to move the stent 46 into the transfer region 38, which simultaneously forces the loading member 50 to slide in the direction A until the stent is contained within the transfer region 38. In this case, the loading member 50 should slide within the transfer region 38 sufficiently easily that when the stent 46 is pulled via the threads 52, the stent 46 itself can force the member 50 from the internal chamber of the transfer region 38 without damaging the stent. In another example in which threads 52 are attached in some way to the loading member 50, either pulling either the threads 52 that extend past the end of the device 30 or pulling the cap 56 of the loading member 50 will simultaneously move the loading member 50 in the direction A and pull the threads 52 and attached stent 46 in the direction A.

To facilitate easier grasping of threads 52 extending from the distal end 44 of the device 30, particularly when such threads 52 are not attached to loading member 50, the threads 52 can optionally be attached to a device such as a ring 60, as shown in FIGS. 2 and 3. Many alternative devices or arrangements may be used in place of the ring 60, such as a bar or some other device that is preferably larger than the interior dimensions of the opening 58 of the loading member 50 to maintain the threads 52 in a position outside the device 30. In this way, the stent 46 can be pulled from the storage region 34 by grasping and pulling the device attached to the threads 52, such as the ring 60, to cause the loading member 50 to retract from the transfer region 38.

Alternatively, a mechanism other than threads or sutures can be used to transport the stent to the transfer region, such as a relatively flexible elongated member with hooks or other means of grasping the stent to pull it into the transfer region. This alternative to the threads would preferably also be relatively thin and elongated so that it can extend from the stent to which it is temporarily or permanently attached and through the end of the device opposite the transfer region thereof. The mechanism would preferably also be thin enough that it can extend through the opening in the body of a loading member, when such a configuration is used. The mechanism used in such a way is preferably also relatively easy to sever or otherwise detach from the stent when desired in the process of loading the stent into a delivery tool and/or for implantation of the stent in a patient.

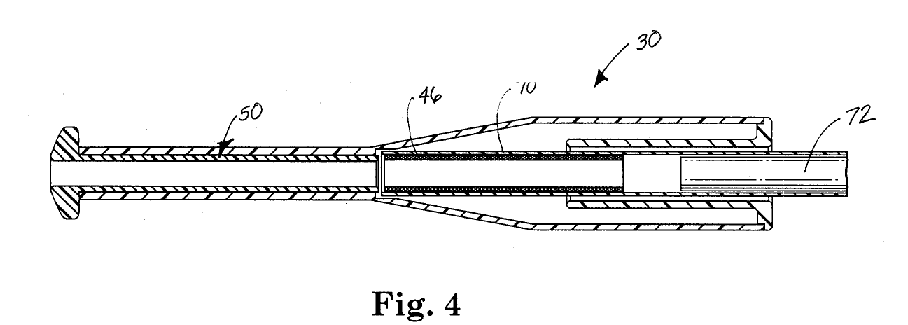

After the stent 46 has been compressed and is being held within the transfer region 38, it is preferable that the stent 46 be transferred to a delivery tool so that it can be placed in a predetermined location within a patient. If the stent 46 is made of a polymeric material, it is further preferable that the stent be compressed and quickly transferred to a delivery tool for relatively immediate deployment thereof. In this way, any plastic deformation of the stent within the transfer region 38 can be minimized or avoided. Referring particularly to FIGS. 3 and 4, once the stent 46 is compressed and contained in the transfer region 38, it can be transferred to or loaded into a wide variety of delivery tools that are used for implantation of the stent in a desired location within a body cavity. In particular, the illustrated portion of the delivery tool shown includes an outer tube 70 and a generally concentrically located inner tube 72, where a distal end 74 of the outer tube 70 is positioned adjacent to the transfer region 38. In particular, the outer tube 70 is inserted into the proximal end 42 of the device 30, through the storage region 34 and the compressing region 36 until it reaches the transfer region 38. While it is preferable that the outer tube 70 of the delivery tool is sized so that it can be inserted through the entire length of the device 30 until it reaches the transfer region 38, the distal end 74 of the outer tube 70 may instead be spaced from the transfer region 38 and the stent 46 contained therein. In any case, it is preferable that the distal end 74 of the outer tube 70 is positioned close enough to the transfer region 38 that the compressed stent 46 can be transferred into the outer tube 70 without substantially expanding upon movement from the transfer region 38, as described in further detail below.

As shown, the inner tube 72 is retracted relative to the outer tube 70 to thereby provide a receiving area 76 adjacent the distal end 74 of the outer tube 70. In particular, the receiving area 76 extends from the distal end 74 of the outer tube 70 to a distal end 78 of the inner tube 72. The receiving area 76 preferably has a sufficient length to receive all or most of the length of the stent to be inserted therein. To move the compressed stent 46 from the transfer region 38 into the receiving area 76 of the delivery tool, the loading member 34 is pushed in a direction B until the stent 46 is completely or substantially contained within the end of the receiving area 76, as shown best in FIG. 4. The device 30 may then be removed from the outer tube 70 so that the compressed stent 46 may be deployed from the delivery tool when desired.

Outer tube 70 is preferably strong enough to maintain a stent being contained therein in its compressed state, yet in some cases, the outer tube 70 is also preferably flexible enough to allow sufficient maneuvering of the delivery tool within a body cavity to implant the stent. When flexibility of the outer tube 70 is desired, the tube 70 may be made of a high strength thermoplastic elastomer such as nylon, PTFE, polyvinylchloride, or the like, for example. Alternatively, if such flexibility of the outer tube 70 is unnecessary or if it desirable to provide an outer tube with additional strength, a more rigid material may be used for the outer tube 70, such as stainless steel, for example.

Inner tube 72 is smaller in diameter than the outer tube 70 and may be formed of the same or a different material than the outer tube 70. It is preferable that the inner tube 72 can slide relatively easily within the outer tube 70 to allow for smooth deployment of the stent when desired. In particular, the outer tube 70 and inner tube 72 are moved relative to each other during stent deployment to reduce the length of the receiving area 76 so that the distal end 78 of the inner tube 72 holds the stent in place while the outer tube 70 retracts to expose the stent. Preferably, the outer tube 70 can be moved a sufficient distance relative to the inner tube 72 that the distal end 74 of the outer tube 70 can move far enough toward the distal end 78 of the inner tube 72 to allow the stent to entirely separate from the outer tube 70. In this way, the receiving area 76 will be eliminated and the entire length of the stent 46 will be released from the distal end 78 of the outer tube 70. However, it is contemplated that the outer tube 70 may remain partially retracted relative to the inner tube 72 during stent deployment so that the final portion of the stent is separated from the outer tube 70 by movement of the outer tube 70 in a direction opposite that of the movement of the stent into the body cavity.

In one preferred embodiment, the inner tube 72 is hollow and is made of a material that has sufficient strength to push the compressed stent from the receiving area 78 of the delivery tool without significant deformation of the inner tube 72. In addition, when the inner tube 72 is hollow, the walls of the tube material are preferably thick enough that a stent being held in the receiving area cannot slip into the interior portion of the inner tube 72 at any point in the stent compression and deployment process. In many cases, it may be desirable for the inner tube 72 to be hollow to allow for the passage of a small device or element (e.g., endoscopes or other viewing equipment, balloon delivery devices to facilitate stent expansion, and the like) through the center of the inner tube 72. A hollow inner tube 72 may further be advantageous if a stent that is not self-expanding is used, wherein a balloon catheter or other device may be inserted through the inner tube 72 and used to expand the stent, when desired. However, if the inner tube 72 is hollow, it may include a cap or end portion (not shown) that covers the distal end 78 of the inner tube 72, or the inner tube 72 could alternatively consist of a solid piece of material. In either of these cases, the likelihood is eliminated of a stent moving into the inner tube 72 at any point during the stent compression and deployment process.

When a device, such as device 30, includes a stent with attached threads, the threads or other extending members can be cut or removed from the stent at any time after the stent is moved into the transfer region, such as immediately after the threads are used to pull the stent into the transfer region of the device. Alternatively, the threads can remain attached to and extending from the compressed stent until the stent is moved from the transfer region into the delivery tool; however, it is preferable that the threads be removed or cut before deployment of the stent by a surgeon. As an alternative to using the threads or sutures attached to a stent, or in conjunction with using threads or sutures attached to a stent to transfer a stent from a storage region to a transfer region, it is contemplated to use a different device to push the stent from the storage region, through the compressing region, and into the transfer region. This could be accomplished, for example, using a relatively flexible material that can compress within the compression region along with the reduction in cross-sectional dimensions of this area.

Referring again to FIGS. 2 through 4, the stent storage region 34 may include a spool insert 80 for retaining the stent in its desired location within the device 30, and also to prevent the stent 46 from compressing or collapsing to a radial dimension that is smaller than that provided by the outside of the spool. In particular, the spool 80 includes a cylindrical support portion 82, a shoulder portion 84, a cap portion 86, and an opening 88 that extends generally through the center of the spool 80 from the cap portion 86 through the support portion 82. The support portion 82 is preferably cylindrical and has an outer diameter that is approximately equal to the inside diameter of the stent 46 in its relaxed state. The support portion 82 is also preferably long enough to support the entire length of the stent 46 in its relaxed position. In this way, no portion of the stent 46 will constrict to a diameter smaller than the outside diameter of the support portion 82 until it is desired to move the stent 46 toward the transfer region 38, in accordance with the present invention. The opening 88 through the spool 80 is preferably cylindrical and large enough to accept the outer tube of a delivery tool that is inserted without requiring excessive force for the insertion. In order to retain the spool 80 within the storage region 34, the shoulder portion 84 preferably has an outer diameter that is slightly smaller than the inside diameter of the proximal end 42 of the device to provide a relatively tight interference fit between these surfaces when the spool 80 is inserted therein. Finally, the cap portion 86 preferably has an outer diameter that is at least slightly larger than the inside diameter of the proximal end 42 of the device 30 to limit the movement of the spool into the storage region 34.

To load a stent 46 into the device 30, the stent 46 is positioned on the support portion 82, which is inserted into the proximal end of the device 30 until the shoulder portion 84 is positioned a sufficient distance into the device 30 that it will not unintentionally be dislodged from the device. Preferably, the shoulder portion 84 is pushed into the interior of the device 30 until the cap portion 86 contacts the distal end 42. The spool 80 is preferably removable and replaceable for reuse of the device 30 with additional stents, if desired. It is understood that the spool 80 could alternatively be omitted from the device 30, that another type of device for supporting a stent could be used, or that a stent may be placed in the storage region 34 without any stent support. In any of these alternatives, a cap may still be placed on the end of the device to keep the stent contained within the device.

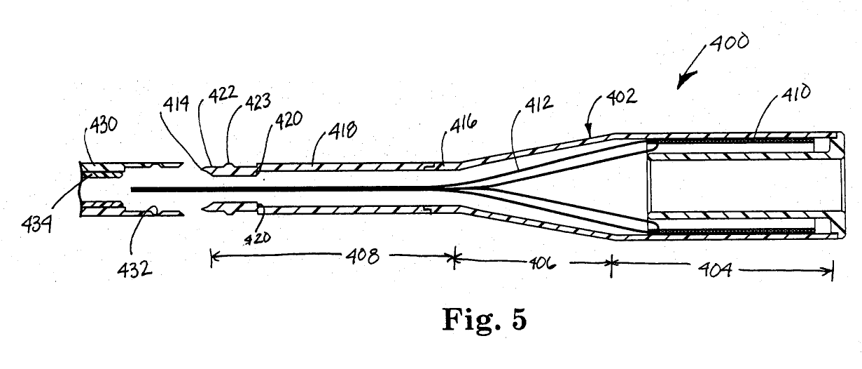

FIG. 5 illustrates another embodiment of a cartridge assembly 400 in accordance with the present invention, which is somewhat similar in structure to the device 30 described above. In particular, assembly 400 includes a cartridge barrel 402 that includes a storage region 404, a compressing region 406, and a transfer region 408. A stent 410 is stored or held within the storage region 404, wherein the stent 410 includes at least one extending member 412, such as a thread, wherein the extending member 412 extends through the length of the barrel 402 and through its distal end 414. In this embodiment, the transfer region 408 of the barrel includes a first portion 416 extending from the compressing region 406 and a second, removable portion 418 that extends from the first portion 416. The removable portion 418 preferably includes a shoulder 420 to stop the compressed stent 410 from being pulled through the distal end 414 of the barrel 402 when the extending member 412 is pulled to move the stent into the transfer region 408. The removable portion 418 further includes a section 422 having a smaller diameter than rest of the portion 418. Section 422 preferably includes at least one protrusion 423 that may extend around part or the entire periphery of the section 422.

To load the stent 410 into a delivery tool, the attached extending member 412 is pulled to move the stent 410 through the storage and compressing regions and into the transfer region 408 until it reaches the shoulder 420. The extending member 412 may then be cut or otherwise removed from the stent 410. The removable portion 418 of the transfer region 408, which is designed to be relatively easily removed from the remainder of the barrel 402, may then be detached from the first portion 416. In one example, the portions 416 and 418 are attached to each other in such a way that turning these portions relative to one another causes allows them to separate. Any other known arrangements of connecting and detaching two tubular sections may be utilized, as desired. In any case, the removable portion 418 will then contain the compressed stent 410 and will be a separate component from the remainder of the cartridge assembly 400.

The removable portion 418 with the compressed stent 410 may then be attached to the end of a delivery tool, a portion of which is shown in FIG. 5. In particular, the end of the delivery tool includes an outer tube 430 having at least one recess 432 that generally corresponds with the protrusion 423 of the section 422, and an inner tube 434. The outer tube 430 is then pressed over the section 422 until the protrusion 423 is positioned within the recess 432, thereby securing the two components to one another. In this embodiment, the stent 410 is then ready for deployment by retracting the outer tube 430 to expose the stent 410 from the end of theremovable portion 418 to a desired location within a body cavity.

Delivery tools used with the stent cartridges of the present invention for stent placement and deployment may have any number of handle configurations, such as those prior art delivery tools having a handle assembly that includes a main body with a fixed rear loop handle and a moveable forward loop handle of the type described, for example, in U.S. Patent Publication No. US 2002/0183827 (Derus et al.), commonly owned by the assignee of the present invention, the entire contents of which are incorporated herein by reference. To deploy a stent from using this type of handle assembly, the surgeon would typically place a thumb in the rear loop handle and a finger in the forward loop handle, then pull the forward loop handle toward the rear loop handle by squeezing the inserted finger toward the thumb. Although it is preferable that the delivery tools used with the stent cartridges of the present invention include a receiving area for the stent within an outer tube of a catheter assembly, it is contemplated that other types of delivery tools that have different configurations for receiving a compressed stent may be used.

In accordance with the present invention, embodiments of handle configurations that are preferably attached to an end of a catheter assembly opposite a stent deployment end are described below. These handle assemblies each involve the use of multiple arms or linkages that can pivot or rotate about each other, with at least one of the arms being provided with a relatively large gripping area for the fingers of a surgeon or other person implanting a stent loaded in the delivery tool. In addition, another arm is preferably spaced at an appropriate distance from the arm having the gripping area so that a surgeon can easily grasp both arms and squeeze them toward each other for precise deployment of a stent. It is further desired with any of the handle assemblies that a stopping device of any appropriate configuration can be used to keep the various handle portions from moving too far from each other before stent deployment. Such a stopping device is preferably designed to provide a retaining area of at least a sufficient size to hold the length of a compressed stent therein. In addition, where rotation points or pivot points are described, it is understood that the movement can be generally unobstructed to allow for smooth rotation, or these points may include a ratcheting mechanism that provides a more “stepped” type of motion for the components relative to each other.

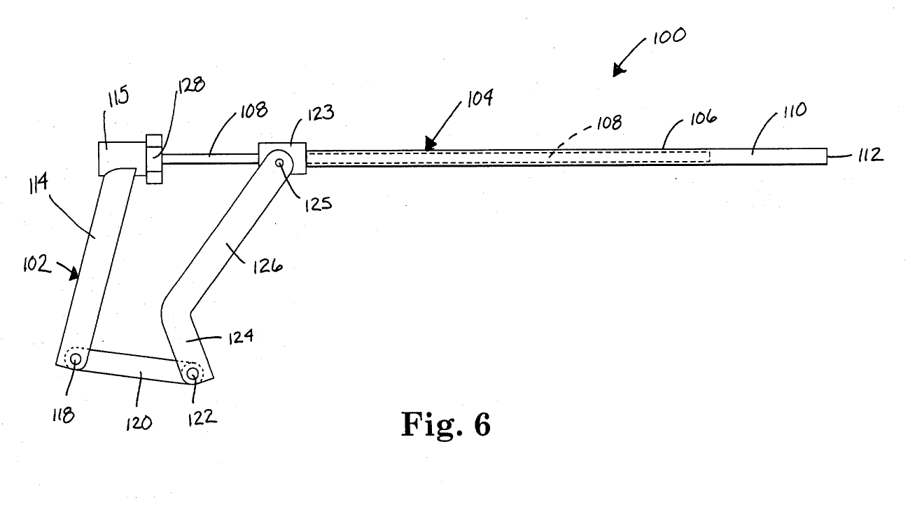

Referring now to FIG. 6, a preferred embodiment of a delivery tool 100 in accordance with the present invention is shown. The delivery tool 100 generally comprises a handle assembly 102 and a catheter assembly 104 extending from the handle assembly 102. The catheter assembly 104 preferably comprises an elongated outer tube 106 and an elongated inner tube 108 slideably positioned within the outer tube 106. The outer tube 106 is preferably at least partially retractable and extendable relative to the inner tube 108 so that a receiving area 110 is provided generally at a distal end 112 of the outer tube 106 for receiving a stent when the outer tube 106 is at least partially extended relative to the inner tube 108. In addition, the outer tube 106 can preferably be retracted or moved a sufficient distance relative to the inner tube 108 to adequately reduce the size of the receiving area 110 to facilitate deployment of the stent.

As described herein, the various tubes of the delivery tool, such as the inner tube 108 and the outer tube 106, may comprise a single tubular piece, or may instead include multiple pieces that are attached to each other but perform functionally as a single unit. The use of multiple pieces for a single tube component may be advantageous, for example, for the inner tube 108 of the delivery tool 100 so that the portion nearest the handle assembly 102 can be provided with greater strength and rigidity than the portion that extends therefrom that will contact the stent. The use of different materials may also be advantageous to minimize the overall weight of the tool, such that pieces or portions that do not require significant strength can be made of a relatively light material.

As shown, the handle assembly 102 includes a first handle portion 114 attached near one end of the inner tube 108. As shown, the inner tube 108 of the catheter assembly 104 is engaged with a receiver 115 of the first handle portion 114. In one embodiment, the inner tube 108 can rotate with respect to the receiver 115 (especially if a locking device is used as described below) but cannot otherwise move with respect to the receiver 115. That is, the inner tube 108 is linearly fixed with respect to the first handle portion 114.

The first handle portion 114 further includes a first pivot point 118 spaced from the receiver 115, at which point a linking arm 120 is pivotally connected to the first handle portion 114. The linking arm 120 is further pivotally connected at a second pivot point 122 to a gripping arm 124. Gripping arm 124 is preferably designed so that a body portion 126 thereof is large enough to accommodate at least two fingers, and preferably four fingers, of a hand of an operator such as a surgeon, and may include indentations or other contours (not shown) for the fingers of the surgeon, if desired. In any case, the gripping arm 124 is arranged so that an end thereof that is spaced from the second pivot point 122 includes a sleeve 123 attached at a third pivot point 125. The sleeve 123 fixedly engages the outer tube 106 of the catheter assembly 104 such that the outer tube 106 cannot rotate or move linearly with respect to the sleeve 123. The sleeve 123 also slideably engages the inner tube 108. As such, the gripping arm 124 can be actuated to translate the outer tube 106 with respect to the inner tube 108 by the linkage comprising the first handle portion 114, linking arm 120, and gripping arm 124.

Optionally, the delivery tool 100 may comprise the ability to lock the inner tube 108 with respect to the outer tube 106 such as is generally desired during insertion of the catheter device 104 into a body lumen for deployment of a stent. For one example, as shown in FIG. 6, the delivery device 100 includes a knob 128 attached to the inner tube 108 for rotating the inner tube 108 with respect to the receiver 115, the sleeve 123, and outer tube 106. Preferably, the sleeve 123 includes a spring-loaded extendable portion such as a pin or the like (not shown) that can engage with a recessed portion of the inner tube 108. For another example, grooves and/or tabs can be provided on various components in the systems to selectively allow and prevent movement of an inner tube with respect to an outer tube. Alternatively, any locking device that locks the inner tube 108 with respect to the outer tube 110, such as a device that locks the handle assembly 102 in place, may be used.

Further referring to FIG. 6, the delivery tool 100 is shown in a position in which the outer tube 106 of the catheter assembly 104 is extended enough to provide a sufficiently large receiving area 110 to accommodate a compressed stent. In this position, the sleeve 123 of the gripping arm 124 is spaced apart from the receiver 115 of the first handle portion 114. When it is desired to deploy the stent, the gripping arm 124 is pulled toward the first handle portion 114, thereby retracting the outer tube 106 relative to the inner tube 108 and reducing the length of the receiving area 110. As this length is further reduced, the stent continues to be pushed or moved from the distal end 112 of the outer tube 106.

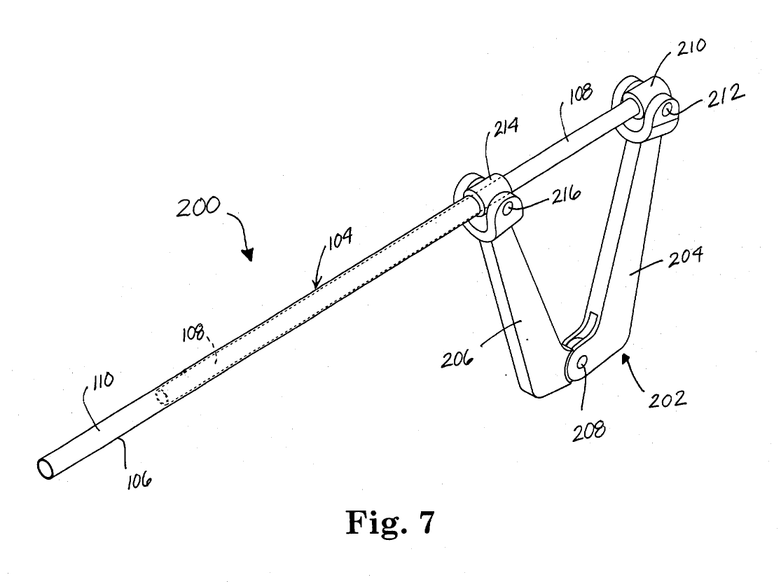

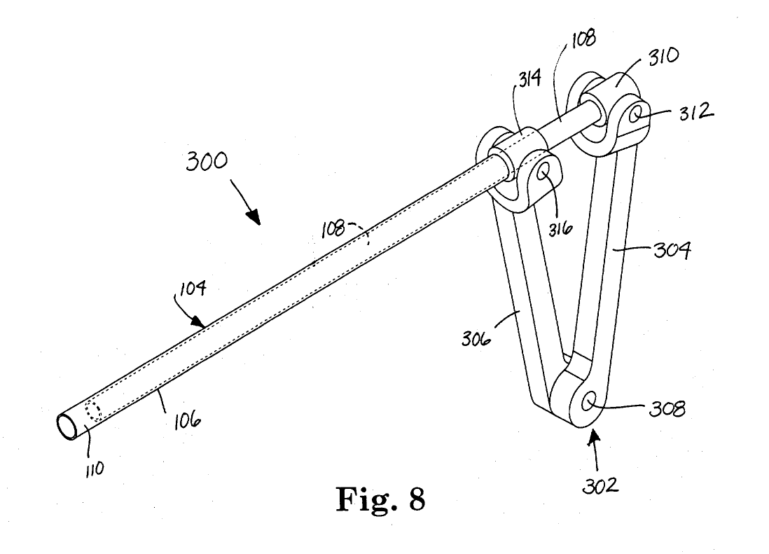

In FIGS. 7 and 8, alternative delivery devices 200 and 300, respectively, are illustrated. As described above with respect to the delivery device 100, delivery devices 200 and 300 each include a catheter device 104 having an outer tube 106 and an inner tube 108. The delivery device 200 includes a handle assembly 202 and the delivery device 300 includes a handle device 302, the particular features of which are explained in further detail below.

The handle assembly 202 comprises a first handle portion 204 and a second handle portion 206. The first handle portion 204 is pivotally attached to the second handle portion 206 at pivot point 208. Preferably, as shown, the first and second handle portions 204 and 206 lie generally in the same plane. The first handle portion 204 includes a receiver 210 for engaging with the catheter device 104 and which is preferably pivotally attached to the first handle portion 204 at pivot point 212. The second handle portion 206 includes a sleeve 214 for slideably engaging with the catheter device 104 and which is pivotally attached to the second handle portion 206 at pivot point 216. As such, the first and second handle portions 204 and 206 may be used to move the inner tube 108 with respect to the outer tube 106 of the catheter assembly 104, to change the length of the receiving area 110.

The handle assembly 302 shown in FIG. 8 is similar to the handle assembly 202 shown in FIG. 7 and includes first and second handle portions 304 and 306. Like the handle assembly 202, the first handle portion 304 includes a receiver 310 attached thereto at pivot point 312 and the second handle portion includes a sleeve 314 attached thereto at pivot point 316. Also, the first and second handle portions 304 and 306 are pivotally attached at pivot point 308. The first and second handle portions 304 and 306 do not lie in the same plane, however. Preferably, the first and second handle portions 304 and 306 are offset with respect to each other. As shown, the first handle portion 304 is generally in the same plane as the catheter device 104 and the second handle portion 306 is offset from the first handle portion 304. As such, the sleeve 314 is offset with respect to the end of the second handle portion 306 such that it is aligned with the receiver 310, as illustrated. Alternatively, if desired, the second handle portion 306 may be in the plane of the catheter device 104 and the first handle portion 304 and the receiver 310 may be offset with respect to the second handle portion 306.

In order to provide the desired movements of the handle portions to deploy a stent from a catheter assembly using handle assemblies of the types described above, it is preferable that each of the assemblies includes at least three pivot points. However, it is understood that actual pivot pins or devices are not necessary, but may instead be replaced by springs or have other configurations that provide the desired degrees of motion for the various components. For example, with a handle assembly like that of assembly 202, it is contemplated that the pivot point 208 about which two separate handle portions 204 and 206 are pivotally connected may instead include a single piece of material that is bent or curved into a generally V-shaped arrangement. In such an arrangement, the two portions on either side of the V-shaped piece can be squeezed toward each other in such a way that the bottom portion of the V-shaped piece can act as a spring to keep the two portions separated from each other until a force is applied to move the two portions toward each other.

While the catheter assemblies are described above as including two tubes that slide relative to each other to deploy a stent, it is understood that the catheter assemblies may include any arrangement of components that would facilitate the deployment of a stent when the components of a handle assembly are manipulated in any of the manners described. For example, more or less than two tubes may be used, or the tubes may not be concentrically arranged relative to each other. Many other variations of the catheter assembly are contemplated and considered to be within the scope of the present invention.

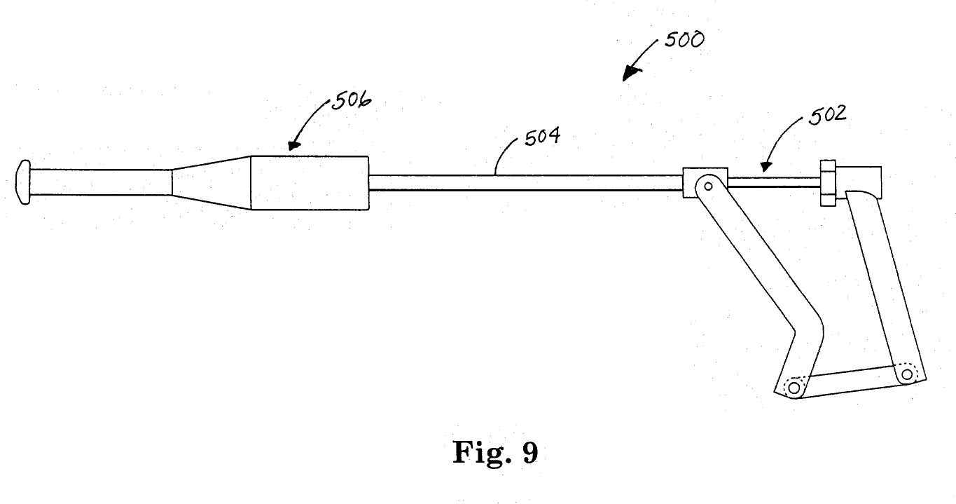

Finally, FIG. 9 illustrates one preferred embodiment of a delivery system 500 in accordance with the present invention, which generally includes a delivery tool 502 having a catheter assembly 504 inserted into one end of a stent cartridge 506. This figure illustrates the arrangement of components relative to each other when a stent has been moved from a transfer region of the stent cartridge and loaded into the end of the catheter assembly 504. At this point, the cartridge 506 may be removed from the catheter assembly 504 and discarded, and the loaded delivery tool 502 may be used to deploy the stent being held within the end of the catheter assembly 504 into a body lumen.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A device for loading a self-expanding stent into a receiving area of a delivery tool, the device comprising:
a stent storage region having an internal chamber sized to retain the stent in a first state;
a stent transfer region spaced from the storage region and having an internal chamber sized to receive, retain, and transfer the self-expanding stent in a second state in which the stent is at least partially compressed relative to the first state of the stent;
a stent compressing region extending between the storage region and the transfer region and having an internal chamber for radially collapsing the stent from the first state to a second state; and
a loading mechanism slideably positioned relative to the transfer region for slideably moving the stent in the second state from the transfer region toward the compressing region.

2. The device of claim 1, further comprising a stent transfer mechanism for moving the stent from the storage region to the transfer region through the compression region while compressing the stent from the first state to the second state.

3. The device of claim 2, wherein the stent transfer mechanism comprises an extending member that is moveable from the storage region to the transfer region.

4. The device of claim 3, wherein the stent transfer mechanism is removably attached to a portion of the stent.

5. The device of claim 3, wherein the extending member of the stent transfer mechanism comprises a generally flexible material.

6. The device of claim 5, wherein the extending member comprises a thread-like material.

7. The device of claim 1, wherein the loading mechanism comprises an extending portion having a distal end that is engageable with a portion of the stent to slideably force the stent in the second state from the transfer region toward the compressing region.

8. The device of claim 1, further comprising a spool insert positioned at least partially within the internal chamber of the storage region, wherein the spool insert is sized to support an interior portion of the stent provided in the first state.

9. The device of claim 8, wherein the spool insert comprises a generally cylindrical body portion and an aperture extending through the body portion, wherein the aperture is sized to accept a portion of the delivery tool.

10. The device of claim 8, wherein the spool insert is removable and replaceable relative to the internal chamber of the storage region.

11. The device of claim 8, wherein the spool insert further comprises an support cylinder extending from a cap portion, wherein the cap portion is sized to generally cover the end of the device adjacent the stent storage region, and wherein the support cylinder is sized to support the stent in the first state.

12. The device of claim 1, wherein the stent compressing region comprises an internal chamber that tapers generally uniformly from the internal chamber of the storage region to the internal chamber of the transfer region.

13. The device of claim 1, wherein the stent compressing region comprises a generally funnel-shaped internal chamber having a first end adjacent the storage region and sized for accepting the stent in the first state and further having a second end adjacent the transfer region and sized for accepting the stent in the second state.

14. The device of claim 1, wherein the loading mechanism further comprises an elongated body portion having a contact end sized and configured to contact a portion of the stent in the second state to move the stent toward the compressing region.

15. The device of claim 14, wherein the loading mechanism is attachable to the stent in the first state for moving the stent from the internal chamber of the storage region toward the transfer region through the compressing region.

16. The device of claim 15, wherein the loading mechanism has an elongated portion and an aperture through the loading portion sized for accepting an extending member of the stent and wherein the loading mechanism is moveable with the extending member for moving the stent into the transfer region.

17. The device of claim 1, further in combination with a self-expanding stent provided in the first state within the internal chamber of the storage region, wherein the stent comprises a substantially tubular, radially expandable body portion.

18. The combination of claim 17, wherein the loading mechanism is further positioned for slideably moving the stent in the second state from the transfer region into the receiving area of the delivery tool.

19. The device of claim 1, further in combination with a delivery tool comprising an outer tube and an inner tube, wherein the outer tube is slideable relative to the inner tube, and further comprising a receiving area inside the outer tube and adjacent a first end of the outer tube.

20. The combination of claim 19, wherein the outer tube of the delivery tool is sized to be inserted into the internal chamber of the storage region and the internal chamber of the compressing region of the device.

21. The combination of claim 20, wherein the internal chamber of the transfer region is sized so that the outer tube of the delivery tool is not insertable into the transfer region.

22. The combination of claim 19, wherein the delivery tool further comprises a handle assembly.

23. The combination of claim 17, further in combination with a delivery tool comprising an outer tube and an inner tube, wherein the outer tube is slideable relative to the inner tube.

24. The combination of claim 23, wherein the delivery tool further comprises a receiving area adjacent a first end of the tool, wherein the receiving area is sized to accept and retain the stent in the second state.

25. The combination of claim 23, wherein the delivery tool further comprises a deployment assembly for causing relative movement between the inner and outer tubes to move the stent in the second state out of the outer tube of the -delivery tool.

26. The combination of claim 22, wherein the handle assembly comprises a first handle portion having first and second ends, a linking arm having first and second ends, and a gripping arm having first and second ends, wherein the first end of the first handle portion is attached to the inner tube, the second end of the first handle portion is rotatably attached to the first end of a linking arm, the second end of the linking arm is rotatably attached to the first end of the gripping arm, and the second end of the gripping arm is rotatably and slideably attached to the inner tube.

27. The combination of claim 22, wherein the handle assembly comprises a first handle portion having first and second ends and a gripping arm having first and second ends, wherein the first end of the first handle portion is rotatably attached to the inner tube, the second end of the first handle portion is rotatably attached to the first end of the gripping arm, and the second end of the gripping arm is rotatably and slideably attached to the inner tube.

28. A device for loading a self-expanding stent into a receiving area of a delivery tool, the device comprising:
a stent storage region having an internal chamber sized to retain the stent in a first state;
a stent transfer region spaced from the storage region and having an internal chamber sized to receive, retain, and transfer the self-expanding state in a second state in which the stent is at least partially compressed relative to the first state of the stent;
a stent compressing region extending between the storage region and the transfer region for radially collapsing the stent from the first state to a second state; and
a loading element slideably positioned within the internal chamber of the stent transfer region and comprising an extending portion for engaging at least a portion of the stent in its second state for slideably moving the stent in the second state from the transfer region toward the compressing region.

29. A delivery system for loading a self-expanding stent into a delivery tool used for positioning and deploying the stent into a predetermined location in a body cavity, the system comprising:
a delivery tool comprising an outer tube having a first end and an inner tube positioned within the outer tube, wherein the outer tube has a first end and a receiving area adjacent to the first end, and wherein the outer tube is slideable relative to the inner tube for receiving and deploying the stent; and
a stent cartridge for providing a stent into the receiving area of the delivery tool, the cartridge comprising:
a stent storage region having an internal chamber sized to retain the stent in a first state;
a stent transfer region spaced from the storage region and having an internal chamber sized to receive, retain, and transfer the self-expanding stent in a second state in which the stent is at least partially compressed relative to the first state of the stent;
a stent compressing region extending between the storage region and the transfer region and having an internal chamber for radially collapsing the stent from the first state to a second state; and
a loading mechanism slideably positioned relative to the transfer region for slideably moving the stent in the second state from the transfer region toward the compressing region.

30. A method of loading a self-expanding stent into the receiving area of a loading tool, the method comprising the steps of:
providing a stent cartridge comprising a stent storage region having an internal chamber including a stent in a first state, a stent transfer region spaced from the storage region and having an internal chamber sized to receive, retain, and transfer the self-expanding stent in a second state in which the stent is at least partially compressed relative to the first state of the stent, a stent compressing region having an internal chamber extending between the storage region and the transfer region, and a loading mechanism slideably positioned relative to the transfer region;
moving the stent from the internal chamber of the storage region through the internal chamber of the compressing region to the internal chamber of the transfer region, wherein the stent is compressed to the second state;
inserting a first end of a delivery tool through an end of the stent storage region and into the stent compressing region, the delivery tool comprising an outer tube having a first end and an inner tube positioned within the outer tube, wherein the outer tube has a receiving area adjacent to the first end, and wherein the inner tube is slideable relative to the outer tube;
transferring the stent in the second state from the transfer region of the cartridge into the receiving area of the delivery tool by moving the loading mechanism toward the compressing region.

31. The method of claim 30, wherein the stent further comprises an elongated transfer member positioned to extend from the storage region to the transfer region.

32. The method of claim 31, wherein the step of moving the stent from the internal chamber of the storage region through the internal chamber of the compressing region to the internal chamber of the transfer region comprises pulling the elongated transfer member to move the stent from the storage region toward the transfer region.

33. The method of claim 32, further comprising the step of removing at least a portion the elongated transfer member from the stent after the stent is moved into the receiving area of the delivery tool.

34. The method of claim 33, wherein the step of removing the elongated transfer member comprises severing at least a portion of the elongated transfer member that extends beyond the first end of the delivery tool.

35. The method of claim 32, wherein the loading mechanism and elongated transfer member simultaneously move in the same direction during the step of moving the stent from the storage region toward the transfer region.

36. The method of claim 32, wherein at least a portion of the loading mechanism extends into the inner chamber of the transfer region prior to the step of moving the stent from the storage region toward the transfer region, and wherein a smaller portion of the loading mechanism extends into the inner chamber of the transfer region after the stent is transferred to the transfer region.

37. The method of claim 30, further comprising the step of removing the delivery tool from the cartridge after the step of transferring the stent from the transfer region of the cartridge into the receiving area of the delivery tool.

38. The method of claim 30, further including step of transferring the stent from the delivery tool to a desired location by moving the outer tube of the delivery tool relative to the inner tube of the delivery tool, wherein the movement of the outer tube relative to the inner tube of the delivery tool reduces the size of the receiving area of the delivery tool.

39. The method of claim 38, wherein the stent expands from the second state toward the first state of the stent during the step of transferring the stent from the delivery tool.
